(19) United States
(12) Patent Application Publication
(10) Pub. No.: US 2025/0134848 A1
(43) Pub. Date: May 1, 2025

(54) METHOD OF ADMINISTRATION OF BRYOSTATIN FOR THE INDUCTION OF TUMOR ASSOCIATED ANTIGENS

(71) Applicant: BryoLogyx Inc., Danville, CA (US)

(72) Inventor: Sathapana Kongsamut, Danville, CA (US)

(21) Appl. No.: 18/834,562

(22) PCT Filed: Jan. 30, 2023
(86) PCT No.: PCT/US2023/011852

§ 371 Date: Jul. 30, 2024

Related U.S. Application Data
(60) Provisional application No. 63/305,198, filed on Jan. 31, 2022.

(51) Int. Cl. A61K 31/35; A61K 45/06

(52) U.S. Cl. CPC A61K 31/35; A61K 45/06

(57) ABSTRACT

Bryostatin-1 stimulates the expression of tumor associated antigens in hematologic and other tumors, enabling these tumors to be recognized by immunotherapies, and increasing clinical responses to various immunotherapies. Accordingly, a method of treating cancer in a subject by administration of bryostatin-1 or functional analogs based on this paradigm is provided herein.

### CROSS-REFERENCE TO RELATED APPLICATIONS

This application claims benefit of priority under 35 U.S.C. § 119 (e) of U.S. Provisional Application No. 63/305,198, filed Jan. 31, 2022. The disclosure of the prior application is considered part of and is herein incorporated by reference in the disclosure of this application in its entirety.

### BACKGROUND OF THE INVENTION

### Field of the Invention

The invention relates generally to cancer and more specifically to induction of tumor associated antigens for improved immune surveillance of aberrant cells in a subject and to provide an enhanced number of targets for immuno-oncology drugs used primarily in the setting of hematologic malignancy.

### Background Information

Cancer is a disease where cells escape normal regulatory processes, transform and grow rapidly. These rapidly-growing cells may arise in blood (hematological malignancy), or as solid tumors in various organs. Abnormal, rapidly-growing cells are distinguished by the expression of unique antigens that are recognized by the host immune system and are held in check by various immune mechanisms. When transformed cells escape normal immune regulatory mechanisms, cells that grow rapidly become malignant tumors, either in blood or as solid tumors.

Such tumors express various antigens termed tumor associated antigens (TAA): cellular proteins constitutively expressed by tumor cells. These antigens provide a stimulus to host immune effector function and in addition serve as targets for immunotherapies, notably monoclonal antibodies or fragments thereof, antibody-drug conjugates (ADCs) and chimeric antigen receptor (CAR)-T cells, and are examples of such immuno-oncology approaches used in hematologic malignancies.

An important aspect of these immune-mediated therapies is the adequate expression of tumor antigens that enable the immunotherapies to recognize aberrant cells for destruction. In B-cell malignancies, TAAs may be specific for tumor types, or may be shared among tumors given their primary origin in B-cells. CD19 and CD20, among other TAAs, are associated with non-Hodgkin's lymphoma, and are the targets of several approved oncology agents. Similarly, other TAAs are associated with chronic lymphocytic leukemia or acute lymphoblastic leukemia (CD22), acute myeloid leukemia (CD33 and CD123) and multiple myeloma (CD38 and B-cell maturation antigen (BCMA)), by way of example. Several studies of these hematologic malignancies have demonstrated that the quantitative expression, or density, of TAAs expressed by tumor cells is an important factor in the efficacy of immunotherapies. Although less common, T-cell malignancies (lymphomas and leukemias, e.g. T-cell acute lymphoblastic leukemia) also express a variety of tumor-associated antigens.

Bryostatin-1 and its analogs are modulators of protein kinase C (PKC), stimulating PKC activity at low concentrations and causing downregulation of PKC at high concentrations. Stimulation of PKC induces the expression of certain genes, including those of tumor associated antigens, and interacts with various intracellular signaling mechanisms. For example, bryostatin-1 has been shown to increase cell-surface expression of CD22, a tumor associated antigen in B-cell tumors such as acute lymphoblastic and chronic lymphocytic leukemia (ALL and CLL) (Varterasian et al., 2000; Biberacher et al., 2012: Ramakrishna et al., 2019).

In earlier studies, bryostatin-1 has been studied as a single agent to treat cancer. These studies used various dosing regimens: notably, long continuous infusion times as long as 3 weeks. The rationale for the prolonged administration was to stimulate cancer cells to differentiate and/or stop growing. In the present invention, short-term administration provides the ‘trigger’ needed to stimulate the expression of tumor antigens. This shorter, ‘trigger dosing’ method will improve the therapeutic index of bryostatin-1 by minimizing the drug exposure needed to stimulate antigen expression to a brief pulse or trigger.

The present invention relates to a method of administering to a subject bryostatin-1 or a functional analog thereof for treating B-cell or T-cell malignancies or initiating, enhancing, or prolonging an anti-tumor response in a subject in need thereof, thereby augmenting the treatment effects on the cancer by initiating, enhancing, or prolonging antitumor responses in the subject to an anticancer agent: in particular, an immunotherapy agent.

The invention further relates to compounds that induce tumor antigen expression, and a method of administration (‘trigger dosing’) of said compounds resulting in stimulation or induction of tumor associated antigen expression. This permits compounds to be administered over short periods (the “trigger”), thereby improving therapeutic index or safety. In theory, short-term administration provides the ‘trigger’ needed to stimulate the expression of tumor antigens. This shorter, ‘trigger dosing’ method will further improve the therapeutic index of bryostatin-1 by minimizing the drug exposure needed to stimulate antigen expression to a brief pulse or trigger.

The particular method of administering bryostatin-1 or a functional analog herein described and claimed involves ‘trigger dosing’, a method of administration where a short pulse of bryostatin-1 or a functional analog causes tumor antigens to be expressed on the cell surface for a much longer period of time, after blood levels of bryostatin-1 or a functional analog are no longer measurable. The particular method of administration involves administering bryostatin-1 or a functional analog thereof by intravenous, intraperitoneal, intramuscular, subcutaneous or oral administration. These methods of administration are designed to give a short pulse or ‘trigger’ to obtain the desired efficacy.

Bryostatin-1 and functional analogs are further known to induce the activation of T cells, a beneficial effect in the treatment of cancer. The particular ‘trigger dosing’ method described here is also efficacious in triggering the activation of T cells.

### SUMMARY OF THE INVENTION

This invention is not limited to the particular compositions, methods, and experimental conditions described, as such compositions, methods, and conditions may vary. Additionally, the terminology used herein is for the purposes of describing particular embodiments only, and is not intended to be limiting, since the scope of the present invention will be limited only in the appended claims.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure.

In one embodiment, the techniques described herein relate to a method of treating cancer in a subject including administering to a subject a bryostatin compound for a time of administration and in a dosage amount effective for inducing upregulation of tumor antigens in the subject, thereby treating cancer in the subject.

In some embodiments of the method described herein, the bryostatin compound is bryostatin-1 or a bryostatin analog. In some embodiments, the time of administration is about 0.5 to 24 hrs. Additionally, in some embodiments the time of administration is about 30 min, about 45 min, about 1 hr, about 2 hrs, or about 3 hrs.

Embodiments herein also include a method further including administering an immunotherapeutic agent. In some embodiments, the immunotherapeutic agent is administered prior to, simultaneously with, and/or subsequent to administration of a bryostatin compound.

In various embodiments of the method described herein, the cancer is selected from the group including hematologic neoplasias, including B-cell lymphomas, T-cell malignancies, multiple myeloma, acute myelogenous leukemia, chronic myelogenous leukemia, myelodysplastic syndrome, acute lymphoblastic leukemia, chronic lymphocytic leukemia, and hairy cell leukemia.

In some embodiments of the method described herein, the immunotherapeutic agent is selected from monoclonal antibodies or fragments thereof, bispecific antibodies, antibody-drug conjugates, chimeric antigen receptor (CAR)-T cells and anti-tumor vaccine products or a combination thereof. In various embodiments, the immunotherapeutic agent binds to a protein selected from the group consisting of CD22, CD19, CD20, CD33, CD37, CD38, CD123, and BCMA.

Additionally, in some embodiments of the method described herein, the method further includes administering to the subject an agent selected from an antibody directed against a tumor associated antigen (TAA), a chemotherapeutic agent, an immunotoxic agent, a vaccine, an immunomodulatory drug, an immune metabolism modifying drug, a targeted therapy, radiation, an anti-angiogenesis agent, CAR-T cell therapy, bispecific antibodies, T cell engagers, an agent that reduces immune-suppression, or a combination thereof.

### BRIEF DESCRIPTION OF THE DRAWINGS

FIG. 1 is a graph that shows increases in expression of CD19 in human peripheral blood mononuclear cells (PBMC), in response to bryostatin-1 (1 nM) given for 45 min, 3 hrs or 24 hrs, showing that a 45 min incubation produces the same elevation as longer incubations.

FIG. 2 is a graph that shows increases in expression of CD22 in human peripheral blood mononuclear cells (PBMC), in response to bryostatin-1 (1 nM) given for 45 min, 3 hrs or 24 hrs, showing that a 45 min incubation produces the same elevation as longer incubations.

FIGS. 3A and 3B are plots that show that the increase in antigen expression remains elevated for 7 days (Ramakrishna, S. et al. (2019) Modulation of target density improves CAR T-cell functionality and persistence. Clinical Cancer Research. 25 (17), 5329-5341).

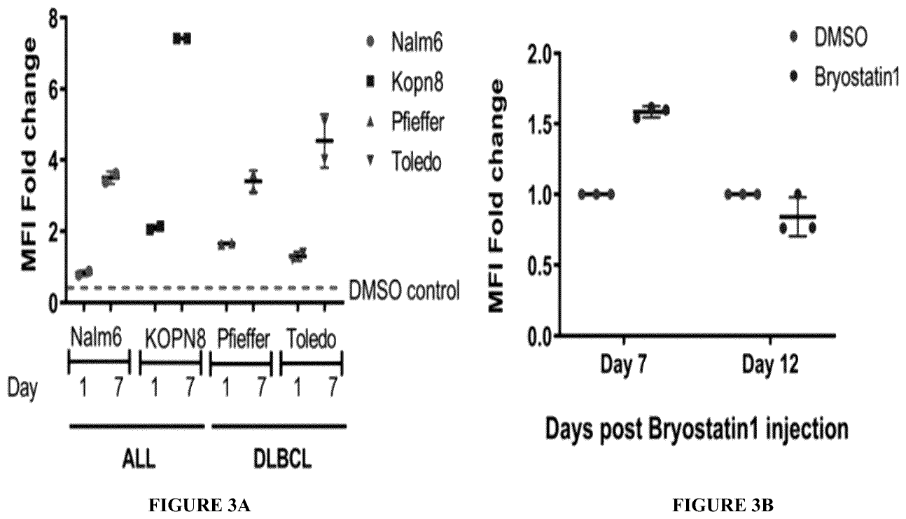

### DETAILED DESCRIPTION OF THE INVENTION

As used in this specification and the appended claims, the singular forms “a”, “an”, and “the” include plural references unless the context clearly dictates otherwise. Thus, for example, references to “the method” include one or more methods and/or steps of the type described herein which will become apparent to those persons skilled in the art upon reading this disclosure.

The terms “subject”, “patient”, or “subjects” as used herein, refer to a human or other mammal, including ungulates, rodents, or primates: for example, humans, horses, cattle, sheep, pigs, goats, llamas, camels, dogs, cats, birds, ferrets, rabbits, squirrels, mice, opossums, lemurs, or rats. In some embodiments, the subject is a human subject.

The term “effective amount” refers to the amount of a compound, composition, or formulation that is sufficient to treat a condition or disease, to produce desirable effects or results, or to reduce, ease, or arrest symptoms of a condition or disease. “Effective amount” is used interchangeably with the term “therapeutically effective amount”.

As used herein, the term “tumor associated antigen” (TAA) is an antigen present in a subject's tumor cell or tissue. TAAs are proteins expressed on the surface of tumor cells that can be recognized by the immune system and immunotherapy agents. Such proteins can be glycoproteins, phosphoproteins, glycophosphoproteins, phosphoglycoproteins and the like. The quantitative expression of the tumor associated antigen (also referred to as the tumor antigen density) is a known correlate of clinical response. These antigens may be expressed in normal cells as well, reflecting their constitutive expression and normal biologic function.

Bryostatin-1 or functional analogs thereof (also known as “bryologs” or bryostatin compounds) refer to a group of macrolide lactones known to be potent modulators of protein kinase C (PKC). Bryostatin-1 is sourced from a marine bryozoan, Bugula neritina, and is a potent modulator of the protein kinase C (PKC) enzyme system, a family of proteins involved in cellular signaling, cell proliferation and cell death. As such, bryostatin-1 has applications across several therapeutic indications. To date, 20 different bryostatin compounds have been isolated. Examples of bryostatin analogs include compounds such as Picolog, acetal 7c and Merle 23. Bryostatin-1 is the best characterized analog, and the only member of the bryostatin family to have been evaluated in human clinical trials. At low concentrations, it is a potent PKC activator, and results in activation of cell signaling, induction of transcription, and the cellular expression of membrane-associated tumor and/or human immunodeficiency virus (HIV) antigens. Bryostatin-1 thus makes these cells more visible to the host immune system, and to exogenously administered immunotherapeutic agents that target these antigens. See for example, Wender et al., U.S. Pat. No. 8,816,122, herein incorporated by reference.

As used herein, the term “treatment” refers to an approach or regimen designed to improve or alleviate symptoms of a disease, sickness, or infirmity. Treatment can lead to reduction in pain, improvement of quality of life, or decrease in size, number, or distribution of a tumor, cancer, or cancerous cells. Treatment of cancer also occurs when symptoms or tests for cancer or cancerous cells are improved, or when symptoms or tests for cancer or cancerous cells do not worsen, or stabilize.

As used herein, the term “bryostatin compound” refers to compounds having an underlying bryostatin pharmacophore, which is based on bryostatin natural products, and in some cases has a scaffold that is characterized by a macrocyclic lactone-containing ring including three embedded six-membered rings (e.g., tetrahydropyran rings designated A, B and C rings), and an arrangement of numbered C1 to C26 carbon atoms, as exemplified in the bryostatin 1 structure shown below. The lactone of the scaffold is defined by a bond between a C1 carbonyl and the oxygen of a C25 hydroxyl group.

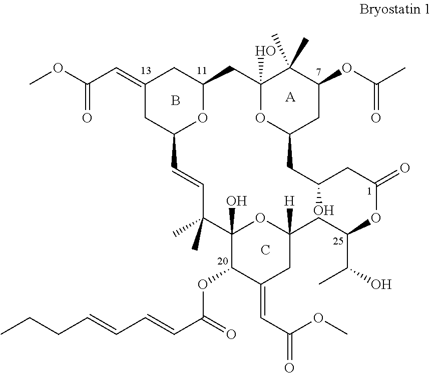

The bryostatin scaffold can include an alkene between C16 and C17 and an exocyclic alkene at positions C13 and C21. The bryostatin scaffold can include a particular arrangement of stereocenters, e.g. at C3, C5, C7, C9, C11, C15, C19, C20 and C23, C25 and/or C26. A variety of substituent groups and derivative groups (e.g., esters or ether groups) can be included in the subject bryostatin compounds (see e.g., Wender et al. WO2018/067382). Naturally occurring bryostatins, originally isolated from a marine bryozoan, include a family of about 21 known compounds. The term “bryostatin compound” is meant to include both naturally occurring bryostatin compounds, such as bryostatin 1, as well as “bryostatin analog compounds”, which include non-naturally occurring bryostatin analogs and derivative compounds of interest that retain functionality required for biological activity.

The term “bryostatin compound” is meant to include a compound having the same underlying bryostatin pharmacophore: namely, a three-dimensional spatial arrangement of three hydrogen bond donors and acceptors that provides its binding function along with a lipid domain that provides for its association with a membrane and for its function derived thereof. This bryostatin pharmacophore, or PKC pharmacophore model, was introduced by the Wender group in 1986 (see e.g., Wender, PNAS, 1986, 83, 4214-4218), and extended to bryostatin in 1988 (see e.g., Wender, PNAS, 1988, 85, 7197-7201), leading to the design of the first bryostatin analogs (e.g., Wender, JACS, 1998, 120, 4534-4535; and Wender, PNAS, 1998, 95, 6624-6629 the disclosures of which are herein incorporated by reference in their entirety). The pharmacophore model is described in U.S. Pat. No. 8,735,609, the disclosure of which is herein incorporated by reference in its entirety.

As used herein, the term “beneficial effect” or “efficacious” refers to an improvement of symptoms of a disease, reduction in detrimental symptoms, improvement of clinical test results, reduction in pain, improvement of quality of life, or other desirable results.

As used herein, the term “administer” refers to any method in which a treatment is given to a patient or subject. Administering can be accomplished via topical, intravenous, intramuscular, systemic, oral, or parenteral methods. Administering can occur at multiple or single points in time and may be combined with multiple treatments.

As used herein, a molecule binding to another molecule refers to the ability of a molecule to target another molecule, whether the molecules are proteins, nucleic acids, antibodies, or small molecules. For example, proteins such as CD19 and CD22 are common antigens targeted by immunotherapies that include antibodies, antibody fragments, diabodies, or other molecules that bind to cancer-related antigens.

The term “upregulation”, as used herein, refers to an increase in the number, percentage, concentration, or amount of antigens presented on the surface of a cancer cell. This could be due to an increase in transcription of the antigens, a decrease in physiological processes that inhibit transcription products from leading to production of the antigen, a decrease in processes that remove antigens from the surface of a cancer cell or tumor, or any other processes that lead to the end result of an increase in antigens presented on the surface of a cancer cell, that then allow the cell to be detected and destroyed by the immune system.

The present invention is based on the finding that bryostatin-1 and analogs thereof can stimulate the expression of TAA, resulting in improved recognition of aberrant (e.g., cancer) cells expressing these antigens, and the improved ability of immune therapies to target and kill these aberrant cells.

Bryostatin-1 stimulates the expression of tumor associated antigens in B-cell tumors (hematologic malignancies), enabling these tumors to be recognized by immune therapies, and increasing clinical responses to various immune therapies. Analogs of bryostatin also stimulate the expression of TAAs in hematologic malignancies, enabling these tumors to be recognized by the immune system and immune therapies. Bryostatin compounds increase clinical responses to various immune therapies. Bryostatin-1 stimulates the expression of TAAs in leukemias, lymphomas and myelomas: specifically, in myelogenous leukemias, non-Hodgkin's lymphoma and multiple myeloma, enabling these tumors to be recognized by immune therapies, improving clinical responses to various immune therapies. Analogs of bryostatin also stimulate the expression of tumor associated antigens in leukemias, lymphomas and myelomas: specifically, in myelogenous leukemias, non-Hodgkin's lymphoma and multiple myeloma, enabling these tumors to be recognized by immune therapies, and increasing the clinical responses to various immune therapies.

A major impediment to the efficacy of cancer immunotherapy relates to the scarcity of TAAs that function to engage host immune function and act as cellular targets for immunotherapeutic agents, including monoclonal antibodies or fragments thereof, ADCs and CAR-T agents. Cell membrane presentation of TAAs, also referred to as TAA density, has been described as a predictor of response to immunotherapeutic agents. Indeed, the clinical failures, or relapses, that occur following CD19 CAR-T in acute lymphoblastic leukemia are associated with the loss and cellular internalization of target CD19 molecules (see for example, Fry et al., 2018). A similar phenomenon occurs following CD22 CAR-T in acute lymphoblastic leukemia, and is associated with the loss and cellular internalization or shedding of the CD22 target molecule (Fry et al., 2018: Guedan & Delgado, 2019).

The ability to minimize side effects is an important parameter in therapeutics: this is particularly true when drugs are given in combination. Hence, reducing the therapeutic exposure of the patient to the minimum dose needed to produce an efficacious result is highly desirable. In a majority of older clinical studies with bryostatin-1, long infusions were employed (see for example: Varterasian et al., 2000, 72 h infusion). For these older studies, the objective was to downregulate protein kinase C and elicit differentiation of tumor cells, thereby stopping tumor growth.

In contrast, the current approach is to stimulate antigen expression so that tumor cells will become visible to cancer immunotherapies, thereby making immunotherapies more effective. Initial animal experiments show that a single intraperitoneal administration of bryostatin-1 is able to induce an elevation of tumor associated antigen expression (Ramakrishna et al., 2019).

There are a number of immuno-oncology drugs and therapies that can be used in combination with bryostatin-1 to treat cancer in patients. Examples of FDA approved antibodies for the treatment of cancer include but are not limited to Avastin (bevacizumab), Bexxar (tositumomab), CDP 870, and CEA-Scan (arcitumomab), denosumab, Erbitux (cetuximab), Herceptin (trastuzumab), Humira (adalimumab), IMC-IIF 8, LeukoScan (sulesomab), Campath (alemtuzumab), MabThera (rituximab), matuzumab, Mylotarg (gemtuzumab ozogamicin), natalizumab, panitumamab, Panorex (edrecolomab), Raptiva (efalizumab), Remicade (infliximab), ReoPro (abciximab), rituximab, Simulect (basiliximab), Synagis (palivizumab), TheraCIM hR3, tocilizumab, Tysabri (natalizumab), Verluma (nofetumomab), Xolair (omalizumab), Zenapax (dacliximab), Zevalin (ibritumomab tiuxetan: IDEC-Y2B8 conjugated to yttrium 90), Gilotrif (afatinib), Lynparza (olaparib), Perjeta (pertuzumab), Opdivo (nivolumab), Bosulif (bosutinib), Cabometyx (cabozantinib), trastuzumabdkst (Ogivri), Sutent (sunitinib malate), Adcetris (brentuximab vedotin), Alecensa (alectinib), Calquence (acalabrutinib), Kymriah (tisagenlecleucel), Yescarta (axicabtagene ciloleucel), Blincyto (blinatumomab), Besponsa (inotuzumab ozogamicin), Lumoxiti (moxetumomab pasudotox-tdfk), LymphoCide (epratuzumab), Verzenio (abemaciclib), Keytruda (pembrolizumab), Aliqopa (copanlisib), Nerlynx (neratinib), Imfinzi (durvalumab), Darzalex (daratumumab), Tecentriq (atezolizumab), Tarceva (erlotinib).

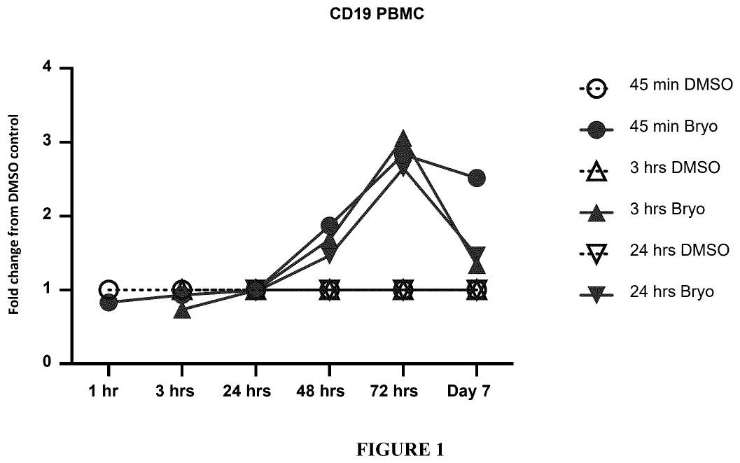

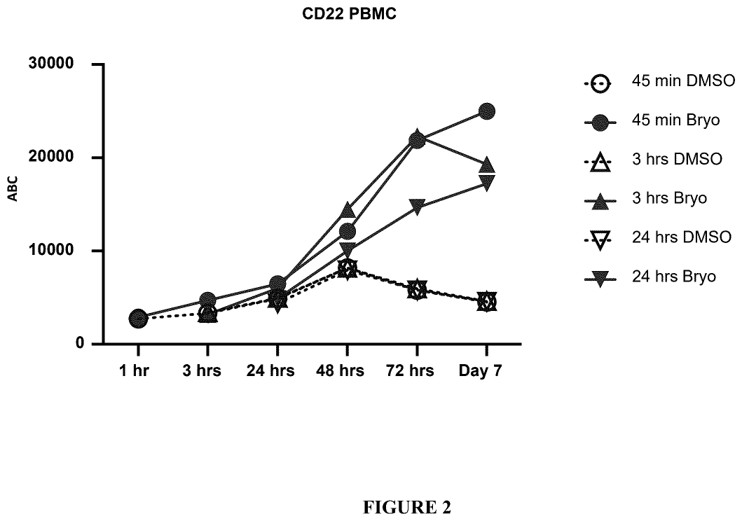

We have shown that short exposure to bryostatin-1 in vitro is sufficient to elevate tumor-associated antigen expression. In experiments performed on numerous tumor cell lines and human cells, bryostatin-1 was administered for varying periods of time, ranging from 45 min to 24 hrs, and changes in the expression of CD19 and CD22 were measured. A short exposure (45 min) gave the same level of increase in antigen expression as 3 h or 24 h exposure (FIGS. 1 and 2). This elevation took time to become evident and was maximal at 72 h. This demonstrates that a short pulse (no longer than 45 min) can elicit a response equal to much longer incubation, corresponding to a short dose rather than a long infusion in a clinical setting.

Given these data, we introduce the concept of ‘trigger dosing’ whereby a short pulse (minutes) of exposure to bryostatin-1 or other agent can elevate tumor antigen expression for days. This method of dosing will reduce the need to subject cancer patients to prolonged exposure to bryostatin-1, and eliminate possible interactions between bryostatin-1 with cancer immunotherapy agents. The ‘trigger’ (bryostatin-1) may be administered at one time (before or after the cancer immunotherapy agent), or more than once (before and after the cancer immunotherapy, and the cancer immunotherapy agent administered at another time, before or after the administration of the trigger. Thus, the administration of the two agents can be separated. This would reduce the potential for drug interactions and adverse events resulting from combination therapy.

In some aspects, the disclosures described herein relate to a method of treating cancer in a subject including administering to a subject a bryostatin compound for a time of administration and in a dosage amount effective for inducing upregulation of tumor antigens in the subject, thereby treating cancer in the subject. In some aspects, the bryostatin compound is bryostatin-1 or a bryostatin analog, either natural or synthetic.

In some embodiments, the time duration of administration is about 15 min to about 24 hrs. Additionally, in some embodiments the time of administration is about 30 min, about 3 hrs, or about 24 hrs. The time of administration can be about 1 min, about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, about 30 min, about 31 min, about 32 min, about 33 min, about 34 min, about 35 min, about 36 min, about 37 min, about 38 min, about 39 min, about 40 min, about 41 min, about 42 min, about 43 min, about 44 min, about 45 min, about 46 min, about 47 min, about 48 min, about 49 min, about 50 min, about 51 min, about 52 min, about 53 min, about 54 min, about 55 min, about 56 min, about 57 min, about 58 min, about 59 min, about 1 hr, about 1.5 hrs, about 2 hrs, about 2.5 hrs, about 3 hrs, about 3.5 hrs, about 4 hrs, about 4.5 hrs, about 5 hrs, about 5.5 hrs, about 6 hrs, about 6.5 hrs, about 7 hrs, about 7.5 hrs, about 8 hrs, about 8.5 hrs, about 9 hrs, about 9.5 hrs, about 10 hrs, about 10.5 hrs, about 11 hrs, about 11.5 hrs, about 12 hrs, about 12.5 hrs, about 13 hrs, about 13.5 hrs, about 14 hrs, about 14.5 hrs, about 15 hrs, about 15.5 hrs, about 16 hrs, about 16.5 hrs, about 17 hrs, about 17.5 hrs, about 18 hrs, about 18.5 hrs, about 19 hrs, about 19.5 hrs, about 20 hrs, about 20.5 hrs, about 21 hrs, about 21.5 hrs, about 22 hrs, about 22.5 hrs, about 23 hrs, about 23.5 hrs, or about 24 hrs.

Administration of a bryostatin compound or bryostatin analog in the methods described herein may be intravenous, intraperitoneal, intramuscular, subcutaneous, oral administration, or a combination thereof. The time duration of administration for a bolus dose or for an oral or subcutaneous dose can vary from that of an intravenous dose, with time durations as short as 10 sec to 3 min, for example.

In some aspects of the methods described herein, the dosage amount of bryostatin compound or bryostatin analog is about 5-60 μg/m². In other aspects of the methods described herein, the dosage amount of bryostatin compound or bryostatin analog is about 30-50 μg/m². In another aspect, the dosage amount of bryostatin compound or bryostatin analog is about 45 μg/m². In various aspects of the methods described herein, the dosage of bryostatin compound or bryostatin analog is about 5 μg/m², about 6 μg/m², about 7 μg/m², about 8 μg/m², about 9 μg/m², about 10 μg/m², about 11 μg/m², about 12 μg/m², about 13 μg/m², about 14 μg/m², about 15 μg/m², about 16 μg/m², about 17 μg/m², about 18 μg/m², about 19 μg/m², about 20 μg/m², about μg/m², about 21 μg/m², about 22 μg/m², about 23 μg/m², about 24 μg/m², about 25 μg/m², about 26 μg/m², about 27 μg/m², about 28 μg/m², about 29 μg/m², about 30 μg/m², about 31 μg/m², about 32 μg/m², about 33 μg/m², about 34 μg/m², about 35 μg/m², about 36 μg/m², about 37 μg/m², about 38 μg/m², about 39 μg/m², about 40 μg/m², about 41 μg/m², about 42 μg/m², about 43 μg/m², about 44 μg/m², about 45 μg/m², about 46 μg/m², about 47 μg/m², about 48 μg/m², about 49 μg/m², about 50 g/m², about 51 μg/m², about 52 μg/m², about 53 μg/m², about 54 μg/m², about 55 μg/m², about 56 μg/m², about 57 μg/m², about 58 μg/m², about 59 μg/m², or about 60 μg/m².

The methods described herein further include administering an immunotherapeutic agent. In some embodiments, the immunotherapeutic agent is administered prior to, simultaneously with, or subsequent to administration of a bryostatin compound.

In various embodiments of the method described herein, the cancer is selected from the group including hematologic neoplasias, including B-cell lymphomas, T-cell malignancies, multiple myeloma, acute myelogenous leukemia, chronic myelogenous leukemia, myelodysplastic syndrome, acute lymphoblastic leukemia, chronic lymphocytic leukemia, and hairy cell leukemia. The cancer can also be urogenital, gynecological, lung, gastrointestinal, head and neck cancer, malignant glioblastoma, malignant mesothelioma, non-metastatic or metastatic breast cancer, malignant melanoma, Merkel Cell Carcinoma or bone and soft tissue sarcomas, haematologic neoplasias, non-small cell lung cancer (NSCLC), breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, or small cell lung cancer.

In some embodiments of the method described herein, the immunotherapeutic agent is selected from monoclonal antibodies or fragments thereof, bispecific antibodies, antibody-drug conjugates, chimeric antigen receptor (CAR)-T cells and anti-tumor vaccine products or a combination thereof. Immunotherapeutic agents include therapies designed to use components of the immune system to target different types of cancers. These include FDA antibodies approved for cancer treatment, checkpoint inhibitors, immunomodulatory agents, cytokines, monoclonal antibodies, cancer vaccines, and chimeric antigen receptor T cells.

Checkpoint inhibitors are immune checkpoint antibodies that work by unleashing the activity of tumor antigen specific T cells by blocking their inhibitory receptor interactions with inhibitory ligands (e.g. PD-1: PD-L1 interactions). In principle, this T cell antigen specificity recognition is universal regardless of the disease type where T cell effector memory is generated to previously encountered tumor antigens. This mechanism is applicable to infectious disease where humans have been previously exposed to viral infection (e.g., CMV) and are able to mount a recall response if they encounter that antigen again. Checkpoint inhibitors for use in the method described herein include but are not limited to ipiliumab (Yervoy™), pembrolizumab (Keytruda™), nivolumab (Opdivo™), cemiplimab (Libtayo™), atezolizumab (Tecentriq™), avelumab (Bavencio™), and durvalumab (Imfinzi™). Cytokines used in cancer treatment for use in the method described herein include interferon alpha, intron A [2b], Alferon [2a], interleukin-2, and aldesleukin. Cancer vaccines for use in the method described herein include Gardasil, talimogene laherparepvec (Imlygic™), and sipuleucel-T (Provenge™). Immunomodulatory drugs for use in the method described herein include, for example, thalidomide, lenalidomide, pomalidomide, and imiquimod.

In various embodiments, the immunotherapeutic agent binds to a protein selected from the group consisting of CD22, CD19, CD20, CD33, CD37, CD38, CD123, and BCMA.

In some embodiments of the method described herein, the method further includes administering to the subject an agent selected from an antibody directed against a tumor associated antigen (TAA), a chemotherapeutic agent, an immunotoxic agent, a vaccine, an immunomodulatory drug, an immune metabolism modifying drug, a targeted therapy, radiation, an anti-angiogenesis agent, CAR-T cell therapy, bispecific antibodies, T cell engagers, an agent that reduces immune-suppression, or a combination thereof.

### Example 1

The ability of bryostatin-1 to promote antigen expression was evaluated in vitro. Human peripheral blood mononuclear cells (PBMC) were cultured according to previously published methods (Ramakrishna et al., 2019). Briefly, cells were washed and then treated with either bryostatin-1 (1 nM) or DMSO (vehicle control) for different times as indicated, ranging from 45 min to 24 hrs. Antibody binding capacity (ABC) was measured by flow cytometry (NCI flow lab); and plotted (FIGS. 1 and 2), either directly or as fold change over DMSO/vehicle control. Data shown in FIGS. 1 and 2 show that 45 min incubation produces the same elevation in antigens as longer incubations.

Although the invention has been described with reference to the above examples, it will be understood that modifications and variations are encompassed within the spirit and scope of the invention. Accordingly, the invention is limited only by the following claims.

What is claimed is:

1. A method of treating cancer in a subject comprising administering to a subject a bryostatin compound in a dosage amount effective for inducing upregulation of tumor antigens in the subject, thereby treating cancer in the subject.

2. The method of claim 1, wherein the bryostatin compound is bryostatin-1 or a bryostatin analog.

3. The method of claim 1, wherein the time for administration is between about 0.5 hrs to 24 hrs.

4. The method of claim 3, wherein the time for administration is about 30 min, about 45 min, about 1 hr, about 2 hrs, or about 3 hrs.

5. The method of claim 1, further comprising administering an immunotherapeutic agent to the subject.

6. The method of claim 5, wherein the immunotherapeutic agent is administered prior to, simultaneously with, and/or subsequently to administration of a bryostatin compound.

7. The method of claim 1, wherein the cancer is selected from the group consisting of hematologic neoplasias, including B-cell lymphomas, T-cell malignancies, multiple myeloma, acute myelogenous leukemia, chronic myelogenous leukemia, myelodysplastic syndrome, acute lymphoblastic leukemia, chronic lymphocytic leukemia, and hairy cell leukemia.

8. The method of claim 5, wherein the immunotherapeutic agent is selected from monoclonal antibodies or fragments thereof, antibody-drug conjugates, bispecific antibodies, chimeric antigen receptor (CAR)-T cells, anti-tumor vaccine products, or a combination thereof.

9. The method of claim 5, wherein the immunotherapeutic agent binds to a protein selected from the group consisting of CD22, CD19, CD20, CD33, CD37, CD38, CD123, and BCMA.

10. The method of claim 1, further comprising administering to the subject an agent selected from an antibody directed against a tumor associated antigen (TAA), a chemotherapeutic agent, an immunotoxic agent, a vaccine, a chemotherapeutic agent, an immunomodulatory drug, an immune metabolism modifying drug, an antibody-drug conjugate a targeted therapy, a bispecific antibody, radiation, an anti-angiogenesis agent, CAR-T cell therapy, a T cell engager, an agent that reduces immune-suppression, or a combination thereof.

11. The method of claim 10, wherein the agent is a bispecific antibody, CAR-T cell therapy, a T cell engager, or a combination thereof.

12. The method of claim 1, wherein the subject is a human.
